(19) United States
(12) Patent Application Publication
(10) Pub. No.: US 2025/0303193 A1
(43) Pub. Date: Oct. 2, 2025

(54) RADIONUCLIDE SOURCE CLIP ATTACHMENT SYSTEM

(71) Applicant: ARTBIO, Inc., Cambridge, MA (US)

(72) Inventors: Gjermund Fjeld Olsen, Fjerdingby (NO); Tanja Aarvak, Oslo (NO); Judit Tjelmeland Oestby, Kraakstad (NO); Olga Salina, Ski (NO); John Emmanuel Aristidou, Cambridge (GB)

(73) Assignee: ARTBIO, Inc., Cambridge, MA (US)

(21) Appl. No.: 19/090,538

(22) Filed: Mar. 26, 2025

Related U.S. Application Data
(60) Provisional application No. 63/571,578, filed on Mar. 29, 2024.

(51) Int. Cl. A61N 5/10; A61B 17/08; A61B 17/10; A61B 17/34

(52) U.S. Cl. CPC A61N 5/1007; A61B 17/083; A61B 17/10; A61B 17/3468; A61N 2005/1019

(57) ABSTRACT

Some aspects of the present disclosure are generally related to a radionuclide source clip handling system configured for use with a radionuclide source holder. In some embodiments, the radionuclide source clip handling system is configured to engage a radionuclide source clip with a radionuclide source holder to maintain a position of a radionuclide source disposed in a receptacle of the radionuclide source holder. Still other aspects are generally directed to related methods of use of the radionuclide source clip and radionuclide source clip handling system.

### CROSS-REFERENCE TO RELATED APPLICATIONS

This Application claims the benefit of priority under 35 U.S.C. § 119(e) of U.S. Application Ser. No. 63/571,578, filed Mar. 29, 2024, the disclosure of which is incorporated herein by reference in its entirety.

### FIELD

Disclosed embodiments are related to a radionuclide source clip handling system, which may be used as a radionuclide source clip attachment system in some embodiments, that is configured for use with a radionuclide source holder, and related methods of use.

### BACKGROUND

Radionuclides such as lead 212 (²¹²Pb) may be used in various applications. Radionuclides such as lead 212 may be formed as a progeny radionuclide in the decay chain of a parent radionuclide, for example, thorium, radon, or radium. Parent radionuclides may be used in any of a variety of generators to generate progeny radionuclides.

### SUMMARY

In some embodiments, a radionuclide source clip handling system comprises a housing including an internal volume; an opening formed in the housing, wherein the opening is configured to receive a distal end portion of a radionuclide source holder extending through the opening into the internal volume; a radionuclide source clip interface disposed in the internal volume of the housing and configured to support a radionuclide source clip thereon, wherein the radionuclide source clip interface is moveable between an unengaged configuration and an engaged configuration, wherein the radionuclide source clip interface is configured to hold the radionuclide source clip spaced from the radionuclide source holder in the unengaged configuration when the radionuclide source holder is at least partially disposed in the opening, wherein the radionuclide source clip interface is configured to engage the radionuclide source clip with the distal end portion of the radionuclide source holder when the radionuclide source clip interface is in the engaged configuration and the radionuclide source holder is at least partially disposed in the opening.

In some embodiments, a radionuclide source holder comprises a housing; a radionuclide source receptacle; a radionuclide source disposed in the radionuclide source receptacle; and a radionuclide source clip disposed on and covering at least a portion of the radionuclide source receptacle, wherein the clip is configured to maintain the radionuclide source in the radionuclide source receptacle.

In some embodiments, a method of attaching a radionuclide source clip interface, comprising: inserting at least a portion of a radionuclide source holder into an internal volume of a housing; moving a radionuclide source clip interface engaged with a radionuclide source clip from an unengaged configuration spaced from the radionuclide source holder to an engaged configuration to engage the radionuclide source clip with the radionuclide source holder; and removing the radionuclide source holder engaged with the radionuclide source clip from the radionuclide source clip interface and the housing.

In some embodiments, a radionuclide source clip includes a clip body and two opposing legs extending away from the clip body. The two opposing legs are configured to be engaged with a portion of a radionuclide source holder and retain the radionuclide source clip on a source receptacle of the radionuclide source holder. The radionuclide source clip also includes a gasket connected to the clip body between the two opposing legs and a protrusion connected to the clip body and disposed between the two opposing legs. The protrusion extends in a direction orientated at least partially away from the clip body.

In some embodiments, a method of handling a radionuclide source includes: attaching a radionuclide source clip to a source receptacle of a radionuclide source holder, where the radionuclide source is disposed in the source receptacle; compressing a gasket of the radionuclide source clip against a portion of the radionuclide source holder surrounding the source receptacle to seal the source receptacle; and maintaining a pose of the radionuclide source in the source receptacle with a protrusion of the radionuclide source clip extending into the source receptacle and in contact with the radionuclide source.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a diagram showing decay pathways between radionuclides, according to some embodiments;

FIG. 2 is an image of a radionuclide handling system including a radionuclide source clip handling system, a transport container, and a radionuclide generator, according to some embodiments;

FIG. 3A is a cross-sectional diagram of a radionuclide source holder in an extended configuration, according to some embodiments;

FIG. 3B is a cross-sectional diagram of a radionuclide source holder in a retracted configuration, according to some embodiments;

FIG. 4A is an exploded view of a radionuclide source clip, according to some embodiments;

FIG. 4B is a perspective view of a radionuclide source clip in an unengaged configuration relative to a radionuclide source holder, according to some embodiments;

FIG. 4C is a perspective view of a radionuclide source clip in an engaged configuration relative to a radionuclide source holder, according to some embodiments;

FIG. 4D is a side view of a radionuclide source clip in an engaged configuration relative to a radionuclide source holder, according to some embodiments;

FIG. 5A is a perspective view of a radionuclide source clip handling system, according to some embodiments;

FIG. 5B is a perspective view of a radionuclide source clip handling system, according to some embodiments;

FIG. 5C is a cutaway view of a plunger and radionuclide source clip interface of a radionuclide source clip handling system, according to some embodiments;

FIG. 5D is a cutaway view of a plunger and radionuclide source clip interface of a radionuclide source clip handling system, according to some embodiments;

FIG. 5E is a perspective view of a plunger and radionuclide source clip interface of a radionuclide source clip handling system, according to some embodiments;

FIG. 5F is a perspective view of a plunger and radionuclide source clip interface of a radionuclide source clip handling system, according to some embodiments;

FIG. 5G is a cross-sectional diagram of a radionuclide source clip handling system in an unengaged configuration, according to some embodiments;

FIG. 5H is a cross-sectional diagram of a radionuclide source clip handling system in an engaged configuration, according to some embodiments;

FIG. 5I is a cross-sectional diagram of a radionuclide source clip handling system retaining a radionuclide source clip, according to some embodiments;

FIG. 6 is a method flow diagram detailing how to use a radionuclide source clip handling system, according to some embodiments;

FIG. 7A is a bottom perspective view of a radionuclide source clip according to some embodiments;

FIG. 7B is a cross-sectional perspective view of the radionucleotide source clip of FIG. 7A;

FIG. 7C is a cross-sectional perspective view of the radionucleotide source clip of FIG. 7A;

FIG. 7D is a cross-sectional perspective view of the radionucleotide source clip of FIG. 7A attached to a radionuclide source holder;

FIG. 7E is a perspective view of the radionucleotide source clip of FIG. 7A where the body is transparent and positioned adjacent to a radionuclide source.

### DETAILED DESCRIPTION

Radionuclides may be used for a variety of applications in such fields as medicine, biology, physics, and other industries. Some radionuclides possess relatively short half-lives and thus may be appropriate for use in various medical applications, such as targeted alpha-particle therapy (TAT) where the relatively short half-lives are preferable for treatment of certain conditions (e.g., any of various cancers such as prostate or carcinoid cancers). The short half-life of these radionucleotides may also limit a patient's time of exposure to the radioactive material and accordingly minimize side effects from the radionuclide treatment. Short half-life radionuclides (e.g., lead 212, ²¹²Pb) may be difficult to ship and/or store because the short half-life may lead to quick decay into undesirable daughter, granddaughter, or other progeny radionuclides during shipping or storage before utilized as desired. In contrast, a parent, grandparent, or other precursor radionuclide to the short half-life radionuclide may have a relatively long half-life, such that the precursor radionuclide may be shipped and/or stored for longer periods without substantively decaying into the undesirable progeny radionuclides. Such precursor radionuclides may accordingly be desirable to generate short half-life radionuclides for applications such as medical treatments. However, the radionuclide source materials for these generators may periodically need to be changed over time as the sources become depleted. In instances where a radionuclide generator is at a location removed from a manufacturing facility, such as a point of use facility or distributed manufacturing network, it may not be desirable or even feasible to ship the entire radionuclide generator to a maintenance facility to replace the radionuclide source.

In view of the above, the Inventors have recognized a desire to facilitate the handling and shipping of radionuclide source materials and systems between different locations such as a manufacturing and/or maintenance facility and a point of use of a radionuclide generator. Some radionuclide generators may include an extendable radionuclide source disposed in a portion of a radionuclide source holder that may be transitioned between an exposed and unexposed configuration (e.g., an extended and retracted configuration) to permit the selective emission of gaseous progeny radionuclides. However, in such constructions the radionuclide source may move within the radionuclide source holder and/or be displaced out of the radionuclide source holder during transport and/or handling of the radionuclide source holder. Thus, in some embodiments, it may be desirable to maintain a pose (i.e., a position and orientation) of the radionuclide source within the radionuclide source holder during transport and/or handling. In some instances, it may also be desirable to seal or otherwise isolate the radionuclide source during such transport and/or handling as well.

In view of the above, the Inventors have recognized the benefits associated with a radionuclide source clip configured to be attached to at least a portion of a radionuclide source holder in which a radionuclide source is disposed. The radionuclide source clip may be configured to maintain a position of the radionuclide source when attached to the radionuclide source holder including, for example, during transport and/or manipulation of the radionuclide source holder. Thus, in some embodiments, the radionuclide source clip may desirably facilitate the transport and handling of a radionuclide source holder containing a radionuclide source disposed therein between different locations (e.g., between a manufacturing and/or maintenance location and a point of use).

In some instances, attaching a radionuclide source clip onto a radionuclide source holder may be difficult due to radionuclide sources typically being handled through shielded gloveboxes, with tongs, and/or via robotic manipulators. Accordingly, the Inventors have further recognized the benefits associated with a radionuclide source clip handling system that facilitates the handling of the radionuclide source clip to the radionuclide source holder. For example, in some embodiments, a radionuclide source clip handling system may include a housing with a radionuclide source clip interface that is configured to hold a radionuclide source clip and is movable between an unengaged and engaged configuration. In some embodiments, a distal end portion of the radionuclide source holder (e.g., including the source receptacle containing the radionuclide source) may be at least partially inserted into the housing of the radionuclide source clip handling system, where the radionuclide source clip interface is configured to move from an unengaged configuration, where a radionuclide source clip that is being held by the radionuclide source clip interface is spaced from the radionuclide source holder, to an engaged configuration, where the radionuclide source clip interface is configured to engage the radionuclide source clip with the radionuclide source holder. Once engaged, the radionuclide source clip may maintain a location of the radionuclide source within the radionuclide source holder and the radionuclide source holder may be removed from the housing of the radionuclide source clip handling system. Advantageously, the radionuclide source clip handling system may be more easily manipulated which may facilitate the use of systems typically used in radioactive material manufacturing processes such as shielded gloveboxes, tongs, robotic manipulators, and/or other systems intended to help remove a user from direct interaction with a source material.

Similar to attaching a radionuclide source clip to a radionuclide source holder described above, it should be further understood that the removal of the radionuclide source clip from the radionuclide source holder may be difficult due to the similar handling constraints associated with manipulation of the radionucleotide source through shielded gloveboxes, with tongs, and/or via robotic manipulators. Thus, removal of the clip may be desirable, in some embodiments, to expose the radionuclide source contained in the radionuclide source holder and to obtain radionuclides for various applications. Accordingly, the radionuclide source clip handling systems described herein may also be used to disengage a radionuclide source clip, for instance, by performing the above-described steps in a reverse manner in some embodiments. In some cases, following the removal of the radionuclide source clip from the radionuclide source holder, the radionuclide source holder may be removed from the housing of the handling system and the radionuclide source clip may be retained within the housing.

The above noted radionuclide source clip may offer several benefits. For example, in some embodiments, when engaged with the radionuclide source holder, the radionuclide source clip may help to maintain the radionuclide source within a corresponding receptacle of the radionuclide source holder the radionuclide source is disposed within. In some embodiments, the radionuclide source clip may also form a seal with the source receptacle. This seal between the radionuclide source clip and the source receptacle may form a sealed volume in which the radionuclide source is retained during transport. This sealed volume may prevent contamination of various surfaces when the radionuclide source is not in use, for example, by preventing any progeny radionuclides from the radionuclide source from being transported outside of the sealed volume and/or depositing on surfaces other than the interior of the source receptacle of the radionuclide source holder and/or the radionuclide source clip.

The various components of the systems described herein, e.g., the housing, the radionuclide source clip handling system, the radionuclide source clip, and/or the radionuclide source holder, may be made of any of a variety of materials that are resistant to radiation from the radionuclide source that may be contained in the radionuclide source holder. Non-limiting examples of materials from which the various components may independently be constructed include metals, ceramics, plastics, polymers, rubbers, combinations of the forgoing, and/or any other appropriate material. However, this list is not exhaustive as other materials are also possible. In some embodiments, the materials of some or all of the components may be at least partially constructed of materials that shield radiation such that they shield at least a portion of radiation that is emitted from a radionuclide source. For example, in some embodiments, the material of the various components may include lead, tungsten, and/or other suitable shielding materials.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

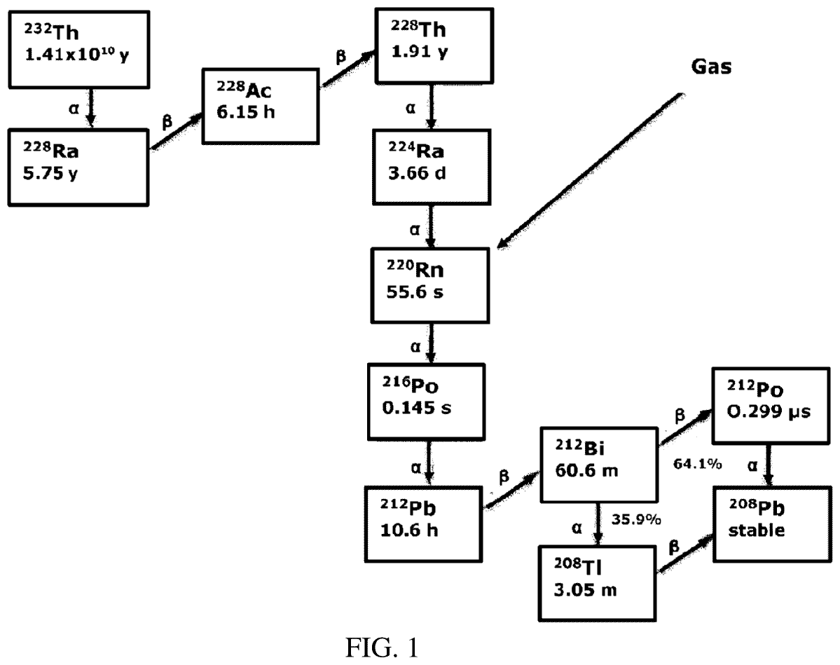

FIG. 1 depicts a diagram of an exemplary thorium series decay chain beginning with thorium 232 (²³²Th). The half-life of each radionuclide in the decay chain is noted in the figure. Note that the radon 220 radionuclide (²²⁰Rn) rapidly decays (e.g., a half-life of 55.6 seconds) into polonium 216 (²¹⁶Po), which further quickly decays (e.g., a half-life of 0.145 seconds) into a lead 212 radionuclide (²¹²Pb). The approximately 10.6 hour half-life of ²¹²Pb may be desirable for various applications. It will further be noted from FIG. 1 that ²²⁰Rn may be a gas at ambient pressure and temperature. Accordingly, it will be appreciated that when a source including a precursor radionuclide to ²²⁰Rn (e.g., ²²⁴Ra, ²²⁸Th, ²²⁸Ac, ²²⁸Ra, or ²³²Th) is exposed to a portion of a container (e.g., an interior surface), the precursor radionuclide may decay into the gaseous ²²⁰Rn. The gaseous ²²⁰Rn may then decay into (e.g., via ²¹⁶Po) ²¹²Pb and may deposit onto the exposed portion of the container as ²¹²Pb.

Note that the materials described in FIG. 1 are exemplary, and that the radionuclide source holder, radionuclide source clip, and radionuclide source clip handling system are suitable for use with any of a variety of radionuclide source materials that may be transported in a radionuclide source holder and/or used to generate progeny radionuclides in a radionuclide generator.

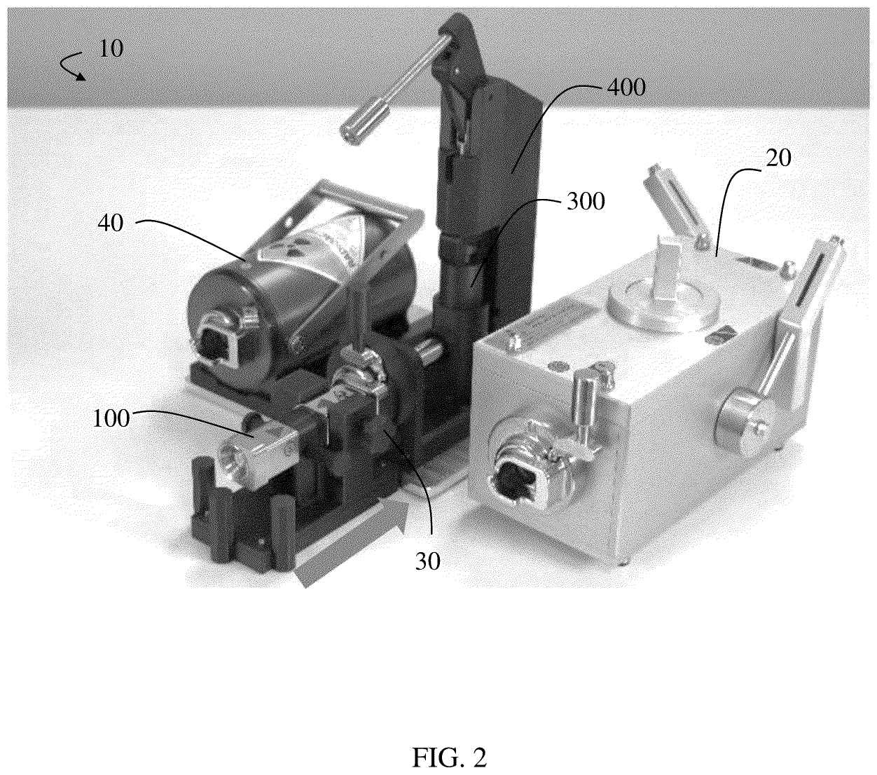

FIG. 2 depicts embodiments of various components of a radionuclide handling system 10. The radionuclide handling system 10 includes a radionuclide generator 20, a support 30, a transport container 40, and a radionuclide source holder 100. The radionuclide source holder 100 is supported in a desired location and orientation by the support 30. The support may include appropriate features that are configured to hold the radionuclide source holder 100 in an appropriate pose such that a portion of the radionuclide source holder 100 may interact with the radionuclide source clip handling system 300 to attach a clip to the radionuclide source holder, as described in more detail elsewhere herein. The system may also include a radionuclide generator 10 that may also be configured to accept the radionuclide source holder 100 for exposing a radionuclide source contained in the radionuclide source holder 100 (e.g., to utilize progeny radionuclides produced therefrom). The radionuclide handling system 10 may also include a radionuclide source transport container 40 that is configured to receive at least a portion of the radionuclide source holder 100 disposed therein for transporting the radionuclide source holder 100

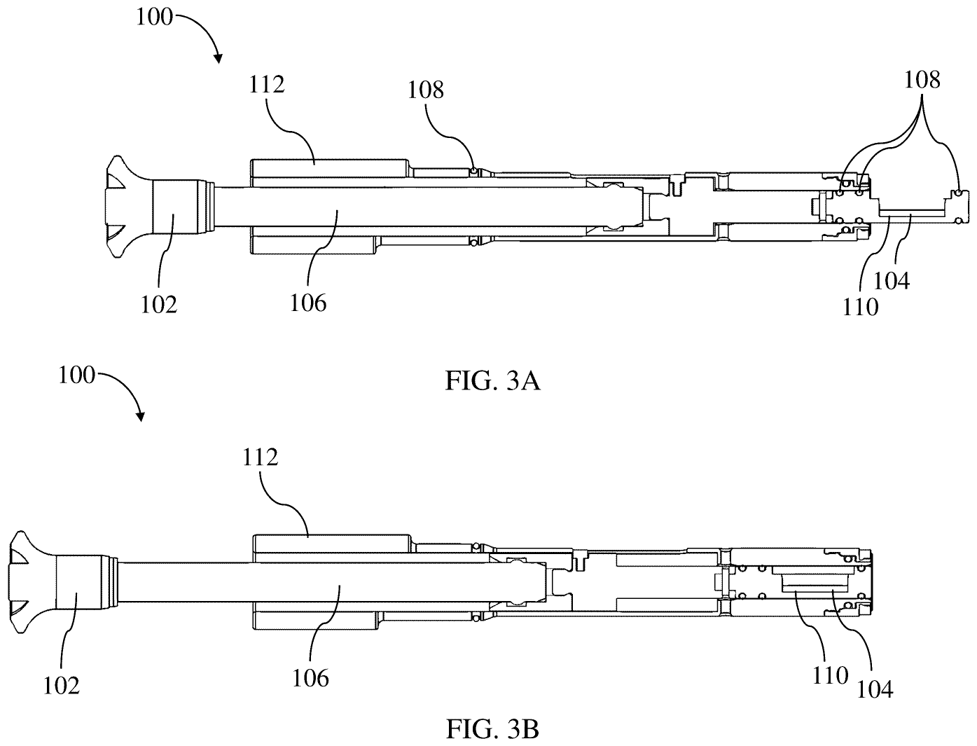

FIGS. 3A and 3B depict cross-sections of the radionuclide source holder 100. FIG. 3A depicts the source 104 in an extended configuration, while FIG. 3B depicts the radionuclide source 104, which may also be referred to as a precursor radionuclide source herein, in a retracted configuration. A radionuclide source holder 100 may include a radionuclide source receptacle 110 configured to receive a radionuclide source 104 disposed therein, for example by including an opening, slot, cavity, divot, channel, or other receptacle sized and shaped to receive the radionuclide source 104 disposed therein. It will be appreciated that a source may be formed in any appropriate regular or irregular geometry, including a cylinder, a disc, a tablet, a block, a chip, a sphere, a sheet, a plate, a ball, a rod, or any other appropriate geometry capable of being positioned in the radionuclide source receptacle 110.

Similarly, although the source receptacle 110 is depicted as a cavity or hollowed-out portion of the source holder 100, it will be appreciated that a source holder 100 may include any source receptacle 110 appropriately formed to receive a correspondingly shaped source. For example, in some embodiments, the source holder may be configured to engage with and retain a source around a perimeter and/or a periphery of the source while opposing sides of the source are exposed. For example, a through hole including two opposing openings may be formed in an exposable portion of a source holder 100 with a source disposed in the through hole. Thus, it should be understood that the currently disclosed systems are not limited to any particular receptacle or source geometry.

As noted previously, the radionuclide source receptacle 110, and the radionuclide source 104 disposed therein, may be moved between an extended and retracted configuration. In the extended configuration the radionuclide source receptacle 110 may be exposed to an exterior environment surrounding the holder 100. Correspondingly, when the radionuclide source holder 100 is in the retracted configuration, the radionuclide source receptacle may be retained within an interior of the radionuclide source holder 100 and optionally isolated from the exterior environment. For example, the radionuclide source receptacle 110 and the source 104 disposed therein may extend out from a housing, such as the distal end portion of the depicted tube, of the holder 100 in the extended configuration. In one such embodiment, the radionuclide source holder 100 may include an elongated housing 112, such as the depicted tube or other structure. The elongated housing 112 may include a channel that extends at least partially, and in some instances completely through the elongated housing. A rod 106 may extend at least partially through the depicted channel of the housing 112 and a distal portion of the rod, or other appropriate linearly translatable component, may be attached to the radionuclide source receptacle 110. Optionally a handle 102, or other structure configured to be grasped or driven in a desired direction, may be connected to a proximal portion of the depicted rod to apply forces to the rod oriented in a direction parallel to the longitudinal axis of the rod to axially move the radionuclide source receptacle 110 between the extended and retracted configurations due to corresponding axial movement of the rod. In the retracted configuration, the radionuclide source receptacle 110 may be retracted into an interior of the holder 100 or otherwise moved to a configuration where it is isolated from an exterior surrounding the holder 100. For instance, in the depicted embodiment shown in FIGS. 3A and 3B, the radionuclide source receptacle 110 is retracted into a distal end portion of the holder when it is in the retracted configuration. Similarly, in the extended configuration, the rod 106 displaces the radionuclide source receptacle 110 out of a distal opening of the housing 112 such that the radionuclide source 104 extends distally from the distal opening of the housing 112.

As noted above, in the retracted configuration shown in FIG. 3B, the radionuclide source 104 may be isolated from a surrounding ambient environment and/or a container attached to or otherwise associated with a radionuclide generator the holder 100 is connected to. In some embodiments, when the source 104 is isolated from the surrounding environment (i.e., is in the retracted configuration), it may be desirable to prevent leakage of gaseous progeny radionuclides from the holder 100. Accordingly, in some embodiments, a radionuclide source holder 100 may include at least one seal 108 that is configured to form a gas-tight interface to isolate the radionuclide source 104 from the surrounding environment when the holder 100 is in the retracted configuration. For example, a radionuclide source holder may include a plurality of seals, each forming a gas-tight interface between the radionuclide source receptacle 110, the rod 106, and/or any other appropriate portion of the source holder 100 with an adjacent portion of the housing 112. For example, one or more seals 108 may be disposed on the radionuclide source receptacle 110 at a location either distally and/or proximally from a portion of the radionuclide source receptacle 110 in which the radionuclide source 104 is disposed. For example, in the depicted embodiment, a single O-ring is disposed on a proximal side of the source 104 to form a gas-tight interface with an internal surface of the housing 112. The depicted embodiment also includes one or more O-rings 108 (e.g., two O-rings as shown in the figure) disposed distally from the source 104 to form a gas-tight seal between the depicted O-rings and the internal surface of the housing 112 when the holder 100 is in the retracted configuration with the illustrated seals 108 in contact with an adjacent portion of the housing 112. It will be appreciated that any number of O-rings or other types of seals may be included at any appropriate location in a radionuclide source holder according to the present disclosure.

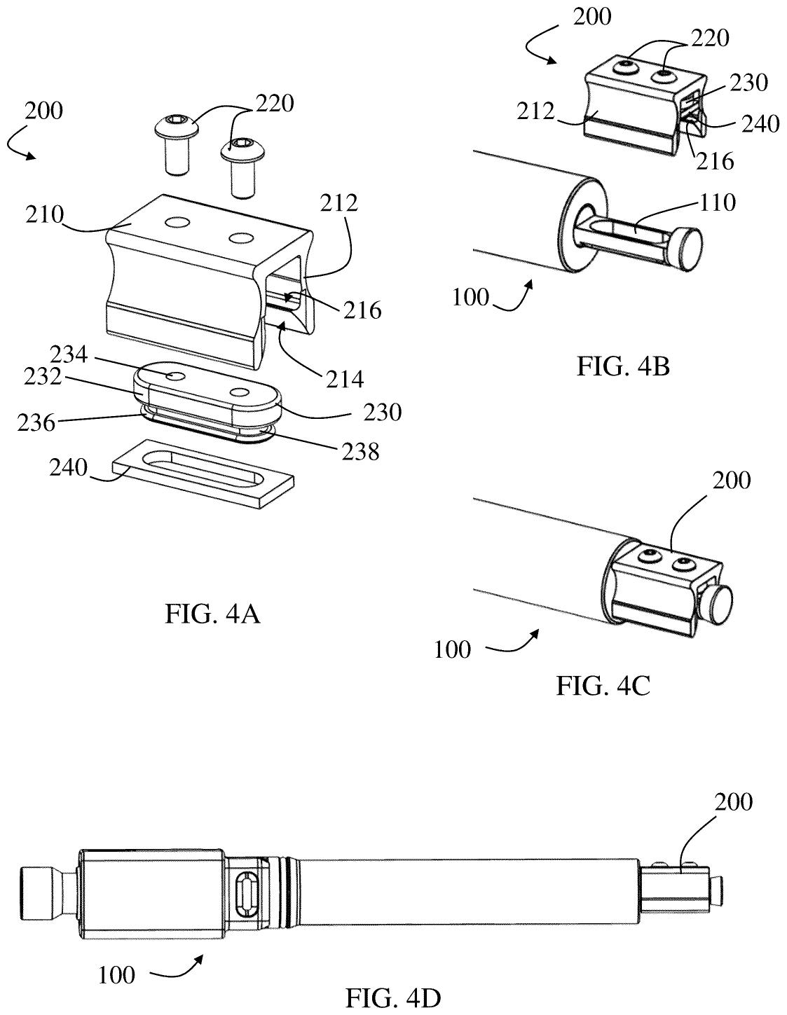

FIG. 4A shows a radionuclide source clip 200, while FIGS. 4B-4D show various views of the radionuclide source clip 200 relative to an extended distal end portion of a radionuclide source holder 100. As shown in the exploded view of the radionuclide source clip 200 in FIG. 4A, the radionuclide source clip 200 may include a body 210, a portion of one or more detents 220, a gasket holder 230, and a gasket 240. In some embodiments, the portions of the detents 220 located on the clip 200 may correspond to threaded fasteners, rivets, spring biased blungers, depressions, and/or any other structures that may function as part of a detent connection to provide a desired retaining force to the clip during operation. Each component of the radionuclide source clip, in some embodiments, may independently be made of materials that are resistant to degradation from exposure to radiation. For instance, each component of the radionuclide source clip may independently be made of metal, plastic, natural or synthetic polymer, or any other appropriate material. In some embodiments, some or all of the components of the radionuclide source clip may include materials that shield radiation.

It should be understood that a radionuclide source clip 200 may be any of a variety of shapes and sizes depending on the construction of the corresponding extendable portion of a radionuclide source holder 100. Regardless the radionuclide source clip 200 may be configured to be attached to the extended portion of the radionuclide source receptacle 110 of the holder 100 to maintain the radionuclide source therein when the clip 200 is attached. Thus, in some embodiments, the radionuclide source clip 200 is sized and shaped to engage with a portion of the radionuclide source holder 100 such as the depicted radionuclide source receptacle 110 extending out from a housing of the holder 100. According to some embodiments, the shape of the radionuclide source clip 200 may be sized and shaped to complement a corresponding portion of the radionuclide source holder 100 to facilitate engagement therebetween while maintaining the source disposed in the radionuclide source receptacle 110 as elaborated on further below. For example, in some embodiments, the radionuclide source clip 200 may be sized and shaped to selectively attach to a portion of the radionuclide source receptacle 110 when it is in the extended configuration and extends distally from a distal end portion of the radionuclide source holder 100.

FIG. 4A shows the body 210 of the radionuclide source clip 200. A pair of opposing legs 212 disposed on either side of a channel extending along a longitudinal length of the clip body 210 may extend away from the clip body 210. The legs 212 may be configured to be engaged with and retain the clip 200 on a portion of the radionuclide source holder 100 such as the radionuclide source receptacle. For example, each of the legs 212 may include a latch including an angled surface 214 and a shelf 216 disposed on a proximal side of the angled surfaces. Thus, the legs 212 may be configured to slide over and past the associated portion of the radionuclide source holder 100 to place the shelves 216 in contact with an associated supporting surface of the radionuclide source holder 100 to prevent relative movement of the clip 200 and the radionuclide source holder 100. For example, when the corresponding portion of the radionuclide source holder 100 is inserted between the legs 212 of the radionuclide source clip 200, the radionuclide source holder 100 presses against the angled surfaces 214 of the legs 212 to bias the legs 212 outwards. This outward movement of the legs 212, allows the radionuclide source holder 100 to move past the angled surfaces 214 and the shelf 216 such that the radionuclide source holder is pressed and held against the gasket of the radionuclide source clip (as shown in FIG. 4C). In such a position, the shelf 216 of each leg 212 of the radionuclide source clip are positioned to retain the clip 200 on the extended distal end portion of the radionuclide source holder 100 which may correspond to the radionuclide source receptacle 110 as elaborated on further below.

As also shown in the figures, the gasket holder 230 may be attached to an internal portion of the clip, for example, between the opposing legs 212 that extend away from the body 210 of the clip 200. The gasket holder 230 may be retained in the channel of the clip 200 at a location between the two legs 212 of the body 210 using any appropriate type of attachment including, for example, interlocking mechanical features, adhesives, welds, threaded fasteners (e.g., the illustrated screws that may function as detent 220 that are engaged with threaded holes 234 formed in a first portion 232 of the gasket holder 230), and/or any other appropriate type of connection to retain the gasket holder 230 in a desired location. The gasket holder 230 may include a second portion 236 that extends towards the radionuclide source holder when the clip 200 is disposed thereon. In some embodiments, the second portion 236 of the gasket holder 230, or other portion of the clip 200, may be configured to extend vertically below the gasket 240 relative to the clip body 210, or other portion of the clip 200, and into the radionuclide source holder receptacle when the clip 200 is attached thereto to maintain a desired position of the radionuclide source. Additionally, the gasket holder 230 may also be configured to retain the gasket 240 thereon using a groove 238 formed on and extending around the gasket holder 230. For example, in the figure, the groove 238 is configured to have a size and shape corresponding to the size and shape of an opening formed in the gasket 240 to hold the gasket 240 on the gasket holder 230 at a desired location within the clip 200 between the legs 212 as elaborated on below. For example, the gasket 240 may be spaced apart from the shelves of the legs to permit the radionuclide source receptacle 110 to be compressed between the gasket 240 and the shelves 216 of the legs 212 of the clip.

It may be desirable to both maintain a position of a radionuclide source within the radionuclide source holder 100 as well as to seal the radionuclide source. Accordingly, in some embodiments, the depicted gasket 240 may be made from a compliant material such that when the gasket 240 is pressed against a surface surrounding the radionuclide source receptacle 110 when the clip 200 is attached to the radionuclide source holder 100, the gasket may form a seal that isolates the radionuclide source from the ambient environment surrounding the holder 100 and clip 200. For example, the gasket 240 may be compressed between the radionuclide source receptacle 110 and the clip body 210. In either case, the gasket 240 and the gasket holder 230 of the radionuclide source holder 100 may facilitate forming a sealed volume with the radionuclide source receptacle 110 in which the radionuclide source 104 may be contained. As noted previously, a source 104 disposed in the radionuclide source receptacle 110 may emit gas. Accordingly, in some embodiments, the gasket 240 may be configured to form a gas-tight scaled volume. Thus, the formation of the sealed volume in which the radionuclide source may be maintained, and may desirably prevent the emission of gaseous radionuclides during handling and transport of the radionuclide source holder 100. It should be understood that the depicted gasket 240 may be made from any appropriate deformable and/or elastically deformable material capable of creating the desired seal as the disclosure is not so limited. Note that, in some embodiments, when the radionuclide source clip is engaged with the radionuclide source holder, there may not be a gas-tight sealed volume.

In addition to the above, in some embodiments, a portion of the gasket holder 230 or other portion of the clip 200 may at least cover, and in some embodiments, extend partially into the radionuclide source receptacle 110 (e.g., the second portion 236 of the gasket holder 230) to maintain a desired position of the radionuclide source within the radionuclide source receptacle 110 when the clip 200 is attached to the holder 100, e.g., in an engaged configuration. For example, FIGS. 4B and 4C show a perspective view of the radionuclide source clip 200 engaging with a distal end portion of the radionuclide source holder 100. The legs 212 of the radionuclide source clip 200 are configured to engage with and maintain the clip 200 on the distal end portion of the radionuclide source holder 100 and to compress the gasket 240 against the radionuclide source receptacle 110 when the clip 200 is positioned thereon. Accordingly, in some such embodiments when a radionuclide source is present in the source receptacle, the attachment of the radionuclide source clip 200 to the radionuclide source holder 100 may facilitate the formation of a sealed volume and/or may maintain a location of a radionuclide source within the radionuclide source receptacle 110. However, it should be understood that in some embodiments, while the radionuclide source clip does not form a sealed volume, it may still physically maintain a position of the radionuclide source material.

While the embodiment depicted in FIGS. 4A-4D show the radionuclide source clip 200 includes two legs 212 with angled clips configured to engage with the radionuclide source holder 100, it should be understood that any number of legs and/or other configurations of a radionuclide source clip 200 configured to be engaged with a radionuclide source holder 100 may be used as the disclosure is not so limited. For example, the radionuclide source clip 200 may be configured to be connected to the radionuclide source holder 100 through any of a variety of connectors. In some embodiments, the radionuclide source clip may engage with the radionuclide source holder via one or more detents, pin-in-hole connections, hook-and-loop connectors, interlocking mechanical features, a magnetic connector, and/or any other appropriate type of connector capable of selectively connecting the radionuclide source clip 200 to the corresponding portion of the radionuclide source holder 100.

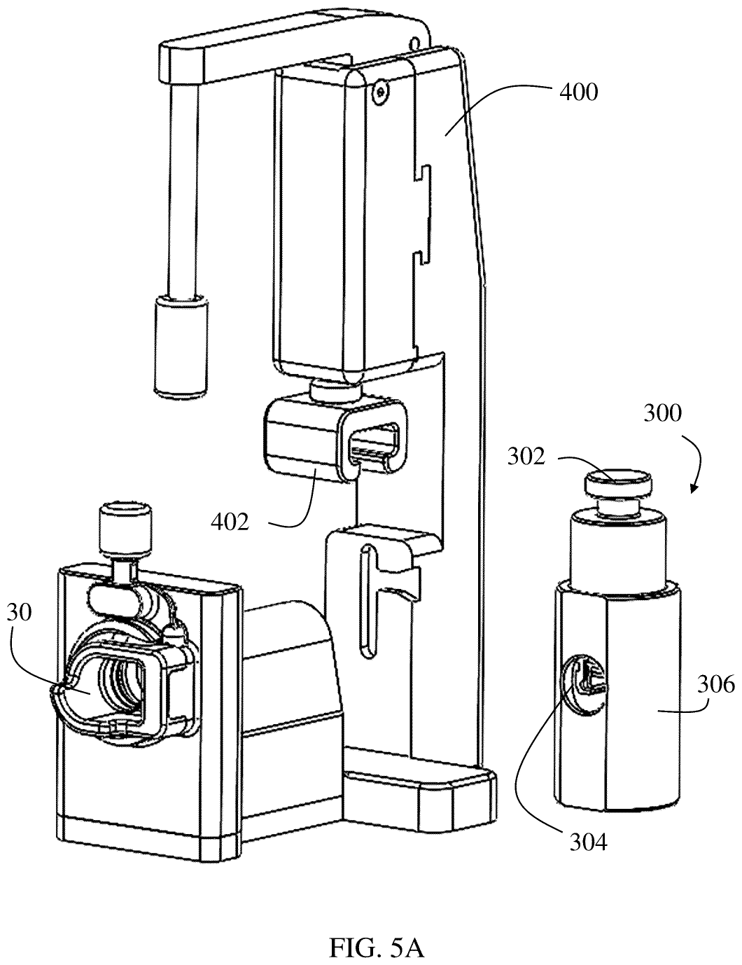

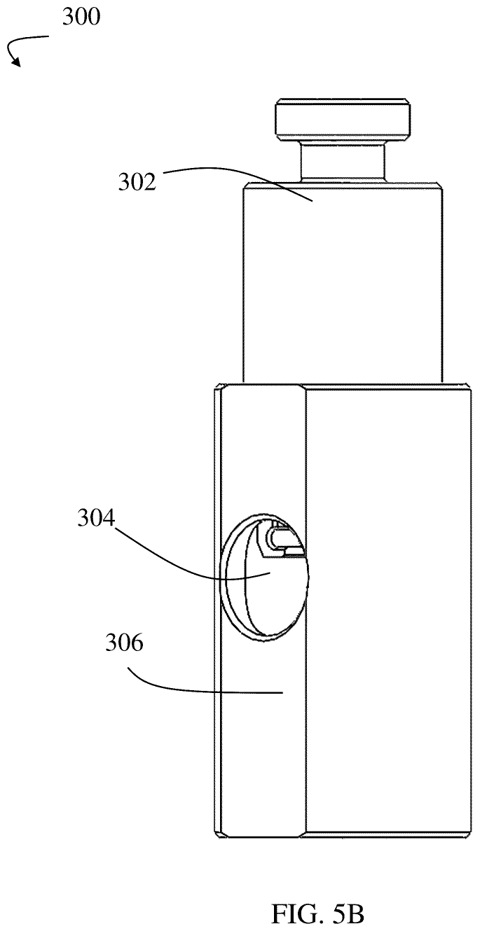

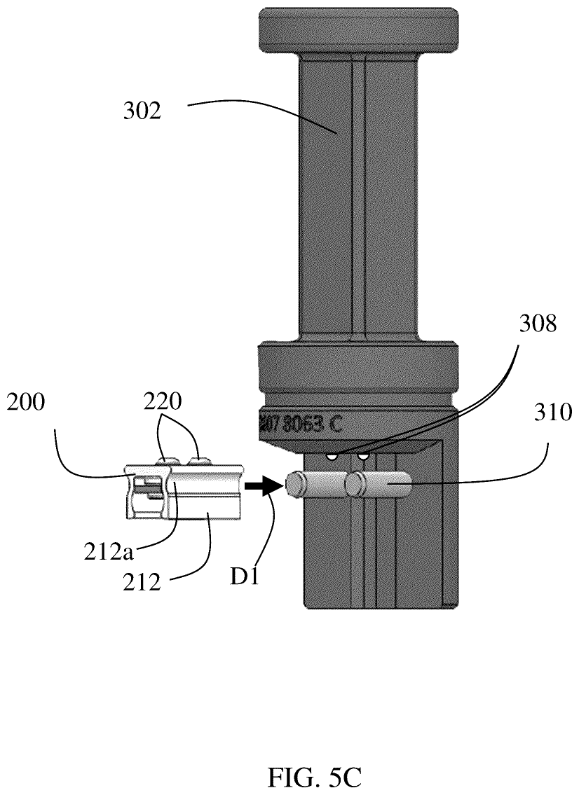

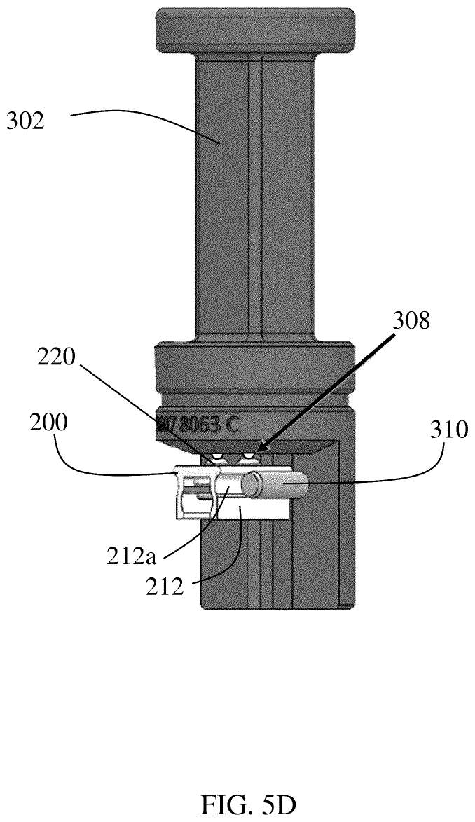

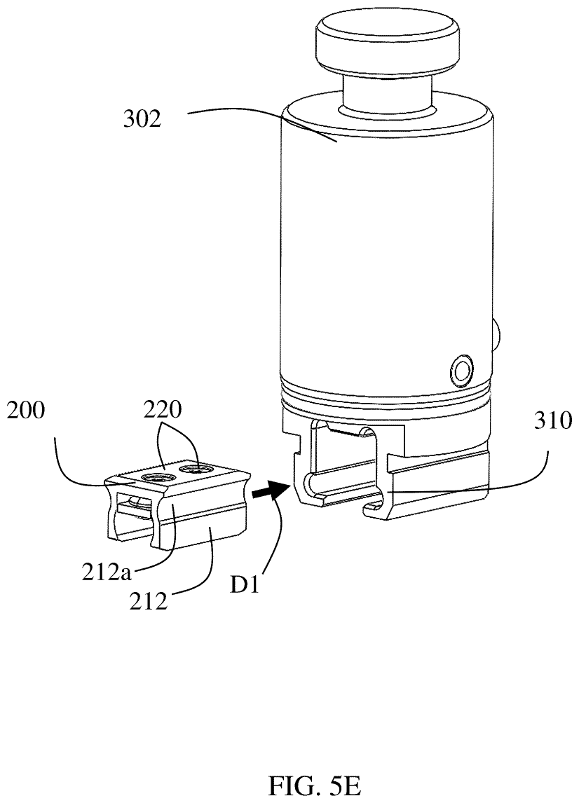

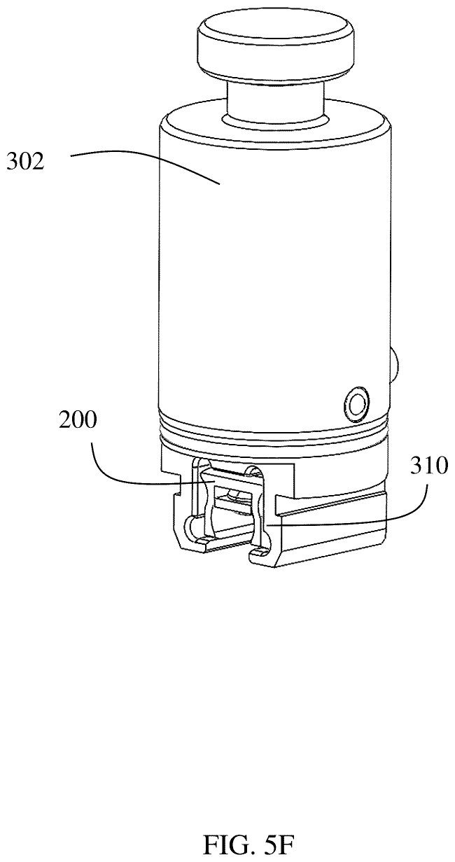

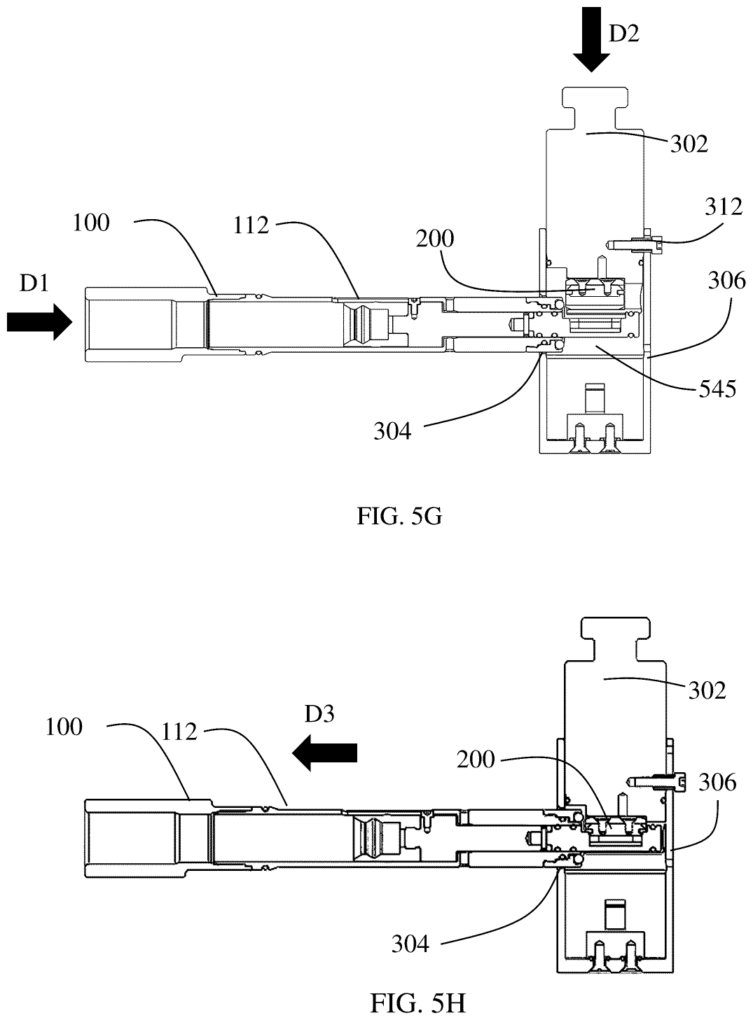

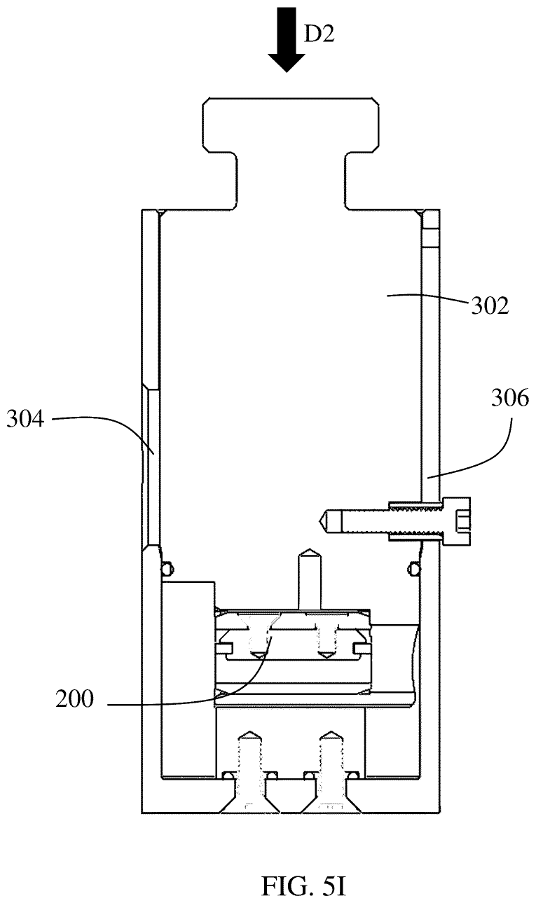

FIGS. 5A-5F show different schematic diagrams of some or all of a system for applying a clip to a radionuclide source holder as described herein. FIG. 5A is a perspective view of an example system for attaching a radionuclide source clip 200 to a radionuclide source holder 100. The system includes a radionuclide source clip handling system 300, a manipulator 400, and a support 30. The radionuclide source clip handling system 300 is configured to be engaged with the manipulator 400 or other appropriate type of actuator where the manipulator 400 may be configured to actuate the radionuclide source clip handling system 300 between an engaged and unengaged configuration to selectively apply a clip to a radionuclide source holder 100 as elaborated on further below. For example, a plunger 302 of the radionuclide source clip handling system 300 may be configured to be engaged within an end effector 402, or other appropriate attachment, of the manipulator 400. For example, mechanically interlocking features of the plunger 302 and end effector may be engaged to permit movement of the end effector 402 of the manipulator 400, or other actuator, to move the plunger 302 in a desired direction. For example, the manipulator 400 may be configured to move the end effector 402 in a vertical direction relative to an underlying supporting surface when a handle, or other trigger, of the manipulator is actuated to displace the connected portion of the plunger 302. This may facilitate the automation and/or manipulation of the system in applications where the system is used in an isolated environment.

During application of a clip, it may be desirable to hold a radionuclide source holder 100, in a desired pose relative to a radionuclide source clip handling system 300. Thus, a support 30 may be configured to receive at least a portion of a radionuclide source holder, e.g., radionuclide source holder 100 as shown in FIG. 2, in a predetermined pose relative to the radionuclide source clip handling system 300 and/or manipulator 400. In some embodiments, the support 30 may be attached to, or otherwise fixed relative to a position, of the manipulator 400. For example, the support 30 may be used to align a distal end portion of the radionuclide source holder 100 with an opening 304 of a housing 306 of the radionuclide source clip handling system 300 when the radionuclide source clip handling system 300 is engaged with the manipulator 400 or other appropriate portion of a system. In some embodiments, the support 30 may include a lock configured to maintain the radionuclide source holder 100 in a desired location and orientation relative to the radionuclide source clip handling system 300 during application of a radionuclide source clip 200 to the radionuclide source holder 100.

FIG. 5B shows an enlarged perspective view of the radionuclide source clip handling system 300. Again, the radionuclide source clip handling system 300 includes a plunger 302, housing 306, and an opening 304 in the housing 306 that provides access to an interior volume of the housing 306. The housing 306 and opening 304 of the radionuclide source clip handling system 300 may be any of a variety of sizes and shapes suitable for insertion of a portion of a holder 100 therein. In some embodiments, the housing 306 of the radionuclide source clip handling system 300 may include an interior volume that is sized and shaped to receive the radionuclide source clip 200 and a distal portion of the radionuclide source holder 100 when the radionuclide source holder 100 is in the extended configuration for attaching a radionuclide source clip thereto as elaborated on further below.

During use, in some embodiments, a distal end portion of a radionuclide source holder 100 may be inserted through the opening 304 such that the distal end portion extends into the internal volume of the housing 306. Accordingly, in some embodiments, a shape and size of the opening 304 in the housing may correspond to a size and shape of the distal end portion of the radionuclide source holder 100 to permit the radionuclide source holder 100 to slide into the opening 304. To help minimize leakage of radioactive material, it may be desirable to form a seal between the portion of the radionuclide source holder 100 inserted into the opening 304 and the housing 306. Thus, in some embodiments, one or more seals (e.g., O-rings, gaskets, etc.) may be configured to form a seal between the housing and a distal end portion of the radionuclide source holder 100 inserted into the housing 306. For example, the seal may help prevent any gaseous progeny radionuclides from the radionuclide source from emanating out of the housing 306 during application of a clip 200 to the radionuclide source holder 100.

As shown in the figures, the plunger 302 is configured to move linearly into and out of the housing 306. For example, according to some embodiments, the plunger 302 may move vertically in and out of the housing relative to a direction of gravity when a base of the housing is disposed on a level supporting surface, though other directions are also possible depending on the construction and orientation of the system. In either case, movement of the plunger 302 may correspondingly move one or more components located internal to the housing 306 of the radionuclide source clip handling system 300. In some embodiments, the plunger 302 facilitates movement of a radionuclide source clip 200 between two or more configurations within the housing 306 of the radionuclide source clip handling system 300 including, for example, moving the radionuclide source clip 200 from an unengaged configuration to an engaged configuration relative to a distal end portion of a radionuclide source holder 100 as elaborated on below.

In some embodiments, and as shown in FIGS. 5C-5D, a plunger 302 includes a radionuclide source clip interface which is configured to support the radionuclide source clip 200 in a desired orientation within the housing and permit the removal of the radionuclide source holder 100 and the radionuclide source clip 200 from of the housing 306 and opening 304 when the holder 100 and clip 200 are in the engaged configuration within the holder 100. For example, radionuclide source clip 200 is configured to slide in the direction of arrow D1 onto and off a radionuclide source clip interface which may correspond to one or more rails 310 in some embodiments. For example, the pair of rails 310 may engage with correspondingly shaped portions of the clip 200, such as the depicted pair of opposing pair of longitudinally extending grooves 212a formed on and extending along the longitudinal length of the opposing pair of legs 212 of the clip 200. The grooves 212a may be sized and shaped such that they may be slid onto and off of the pair of rails 310. This may help to maintain a desired alignment of the radionuclide source clip within the housing 306. For example, a longitudinal axis of the clip 200 and/or a direction of movement along the rails 310 may be parallel to a direction of insertion of a radionuclide source holder 100 into and out of the opening 304 of housing 306.

FIGS. 5E and 5F show an alternative embodiment to that shown in FIGS. 5C and 5D which depicts a plunger 302 including a radionuclide source clip interface which is configured to receive and support the radionuclide source clip 200 in a desired orientation within the housing 306. The embodiment shown in FIGS. 5E and 5F similarly permit the removal of the radionuclide source holder 100 and the radionuclide source clip 200 from of the housing 306 and opening 304 when the holder 100 and clip 200 are in the engaged configuration within the holder 100. For example, radionuclide source clip 200 is configured to slide in the direction of arrow D1 into and out of a radionuclide source clip interface which corresponds to a housing 311 including a cavity sized and shaped to compliment a portion of an exterior of the clip 200 such that it may receive and retain the clip 200 within the cavity. Thus, the clip 200 may be sized and shaped such that it may be slid into and out of the housing 311. The corresponding size and shapes of the radionuclide source clip interface and the radionuclide source clip 200 may help to maintain a desired alignment of the radionuclide source clip within the housing 306. For example, a longitudinal axis of the clip 200 and/or a direction of movement into the housing 311 may be parallel to a direction of insertion of a radionuclide source holder 100 into and out of the opening 304 of housing 306.

Of course, other types of supporting arrangements not shown in FIGS. 5C-5F that are configured to receive the clip 200, configured to provide support for the clip 200 in a first direction (e.g., vertical), and permit the removal of the clip 200 in a second direction (e.g., horizontal) may also be used.

Again, referring to FIGS. 5C and 5D, in some embodiments, a radionuclide source clip interface may include a portion of one or more detents 308 such as spring-loaded plungers, spring loaded balls, elastic components, a cantilever beam with engaging features, or other appropriate type of detent configured to engage with a corresponding portion of a detent 220 formed on the clip 200. For example, the clip 200 may include screws, bolts, divots, or other appropriate corresponding portions of a detent 220 that may be configured to be engaged with the corresponding portions of the one or more detents 308 associated with the plunger 302 when the clip 200 is supported on the rails 310. In either case, the one or more detents 308 may apply a retaining force to the radionuclide source clip 200 that resists movement of the clip 200 along the rails 310 until a threshold force greater than the retaining force is applied to the clip 200 in a direction parallel to a direction of movement of the clip 200 along the rails 310 or other supporting arrangement formed in the radionuclide source clip interface. The retaining force may help maintain a desired position of the clip 200 along the rails 310, thereby facilitating a reproducible positioning of the clip 200 within the interior volume of the housing 306 of the radionuclide source clip handling system 300.

FIGS. 5G and 5H show cross sectional images of the radionuclide source clip handling system 300 when attaching a radionuclide source clip 200 to a distal end portion of the radionuclide source holder 100 at least partially disposed in an opening 304 of a housing 306 of the system 300. As shown, a distal end portion of a radionuclide source holder 100 is inserted into the internal volume of the housing 306 through the opening 304 with the radionuclide source holder 100 in the extended configuration with the radionuclide source receptacle extending distally out from housing 112 of the holder 100. FIG. 5G depicts the radionuclide source clip handling system 300 before attaching a radionuclide source clip 200 to the extended distal end portion of the radionuclide source holder 100. In the depicted configuration in FIG. 5G, the radionuclide source clip 200 is selectively attached to the radionuclide source clip interface of the handling system. In this case, the radionuclide source clip handling system 300 is in an unengaged configuration where the radionuclide source clip 200 is positioned within an interior volume of the housing 306 of the handling system 300 in a desired orientation relative to and spaced apart from the distal end portion of the radionuclide source holder 100 within the housing 306 of the handling system 300. The spacing between the radionuclide source clip 200 and the distal end portion of the radionuclide source holder 100 may be any of a variety of distances such that the distal end portion of the radionuclide source holder may be inserted into the housing and the clip may then be moved from the unengaged configuration where it is spaced apart from the holder 100 as shown in FIG. 5G to an engaged configuration as shown in FIG. 5H where the clip 200 is engaged with the extended distal end portion of the holder 100, as detailed below. For example, the plunger 302 may be moved into the housing to move the clip from the unengaged configuration where it is spaced from the adjacent portion of the radionuclide source holder 100 into engagement with the adjacent portion of the radionuclide source holder 100. In some embodiments, the radionuclide source clip 200 may be engaged with the radionuclide source clip interface 310, and the radionuclide source clip interface 310 may be configured to engage the clip 200 with a distal end portion of the radionuclide source holder 100 when the holder 100 is at least partially disposed in the opening 304 of the housing 306 and the radionuclide source clip interface 310 is moved to the engaged configuration.

Again, as noted above, the size and shape of the opening of the housing may be selected to correspond to a size and shape of the radionuclide source holder with minimal clearance. In some such embodiments, the opening of the housing and a portion of the plunger 302 may facilitate alignment of the radionuclide source holder within the radionuclide source clip handling system. For example, the housing 306, the plunger 302, or other appropriate component may be configured to limit an insertion distance of the radionuclide source holder 100 into the housing 306 which may help provide a reproducible alignment between the radionuclide source clip 200 and the corresponding portion of to the holder 100 during use.

In some embodiments, it may be desirable to prevent inadvertent movement of the plunger 302, or other motion stage used to selectively move a radionuclide source clip 200 within housing 306, when the system is in the unengaged configuration. Accordingly, the radionuclide source clip handling system 300 may include one or more detents 312 configured to maintain a position of the plunger 302, or other motion stage, until an applied force is greater than a threshold force. Any appropriate type of detent, including those disclosed elsewhere herein, may be used as the disclosure is not so limited. Alternatively, one or more locks or other structures may be used to selectively permit or prevent movement of the plunger or other actuatable structure of the system, though embodiments in which a detent or other lock is not used are also contemplated.

As noted previously, the radionuclide source clip interface, such as the illustrated rails 310, may be configured to slidingly support the radionuclide source clip disposed thereon such that the clip 200 may linearly translate within the internal volume 545 of the housing 306 of the clip handling system 300 between the unengaged configuration and the engaged configuration. Additionally, by depressing plunger 302 in the direction of D1, the plunger 302 may be displaced into the housing and the radionuclide source clip interface and the clip 200 disposed thereon may be moved from the unengaged configuration shown in FIG. 5G to the engaged configuration shown in FIG. 5H. For instance, depressing the plunger 302 into the housing 306 changes the configuration of the radionuclide source clip handling system 300 from the unengaged to engaged configuration, which proceeds through translational motion of the plunger 302 and radionuclide source clip 200 towards an adjacent distal portion of the radionuclide source holder 100 in the extended configuration. This may attach the clip 200 to the extended distal end portion of the radionuclide source holder 100, such as the radionuclide source receptacle 110 discussed previously above (e.g., deformation and engagement of the legs 212 with the corresponding portion of the holder 100). Note that alternative types of movement are also possible, for example, rotational motion or a combination of translational and rotational motion may be used. In some embodiments, the type of motion and configuration of the radionuclide source clip interface may be selected based on the size and shape of the corresponding mating portions of a radionuclide source holder 100 and radionuclide source clip 200. In either case, once engaged, the clip 200 attached to the radionuclide source holder 100 may be withdrawn from the housing 306 with the radionuclide source holder 100 though the opening 304 once a force sufficient to overcome the retaining force applied by the one or more detents 308 to the clip 200 is applied to the radionuclide source holder 100.

While the above embodiments of a radionuclide source clip handling system have been described above relative to attaching a clip to a radionuclide source holder, in some embodiments, the radionuclide source clip handling systems 300 disclosed herein may be used to remove the radionuclide source clip 200 from the radionuclide source holder 100 by performing the reverse of the above described methods. For instance, when a radionuclide source holder 100 is in the extended configuration and inserted into the interior of the housing 306, the system may be used to attach a clip 200 to the radionuclide source clip interface 310 when it is aligned with the opening of the housing 306 similar to that shown in FIG. 5H. In some such embodiments, the radionuclide source clip interface 310 and the attached clip 200 may then be moved to the disengaged configuration by linearly translating the plunger 302 to the configuration shown in FIG. 5G to expose a radionuclide source disposed in the radionuclide source holder 100. The radionuclide source holder 100 may then be changed from an extended configuration where the radionuclide source is exposed to a retracted configuration, whereafter the radionuclide source holder may then be withdrawn from the housing 306. Thus, the disclosed systems and methods may be used to both attach and remove a radionuclide source clip from a radionuclide source holder.

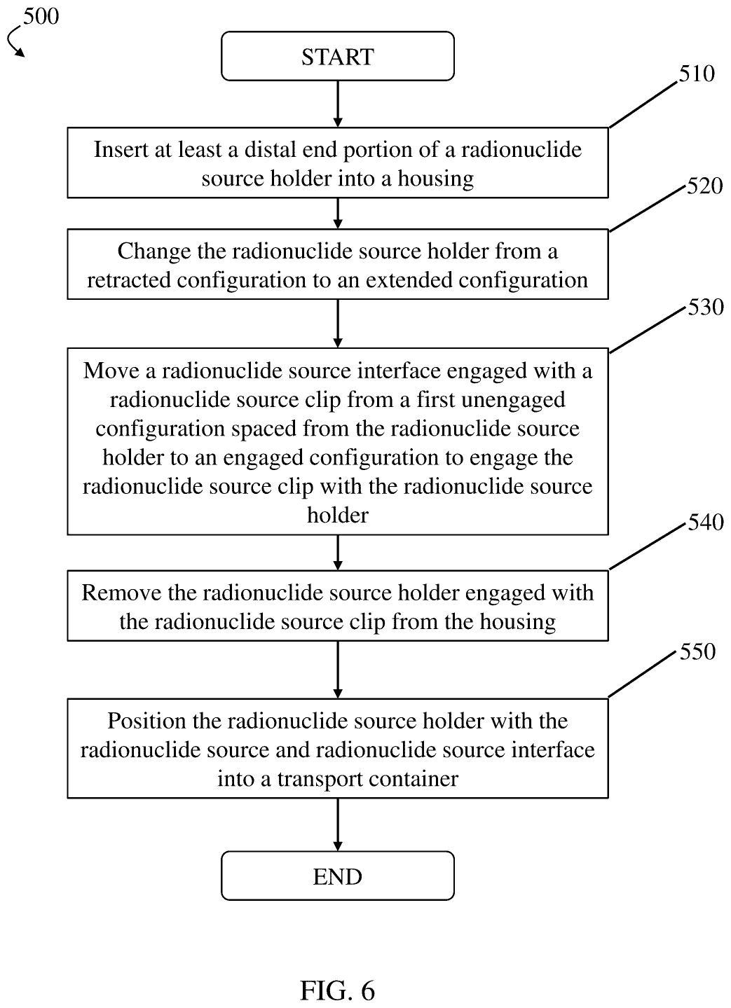

FIG. 6 is a method flow diagram detailing how to use any of the embodiments of a radionuclide source clip handling system described herein. In some embodiments, the method may include inserting a radionuclide source clip onto a radionuclide source clip interface within a housing in an insertion direction D1. The method 500 may include inserting at least a portion of a distal end portion of a radionuclide source holder into a housing at 510. In some embodiments, the portion of the distal end portion of the radionuclide source holder that is inserted into the housing includes a receptacle in which a radionuclide source is contained, e.g., to facilitate selective attachment of the radionuclide source clip thereon. The radionuclide source holder may be supported by a support configured to support the holder in a desired position and orientation relative to the clip during attachment.

The method 500 may further include changing the radionuclide source holder from a retracted configuration to an extended configuration at 520. Changing the configuration of the radionuclide source holder may expose a radionuclide source to an ambient atmosphere and position a desired distal end portion of the holder, such as a radionuclide source receptacle, adjacent to and in a path of movement of the clip. In some embodiments, it is desirable to change the configuration of the radionuclide source holder after inserting it into the housing so that the radionuclide source is contained within the interior volume of the housing, which, in some embodiments, includes materials that are configured to shield radiation.

In some embodiments, the method 500 further includes moving a radionuclide source clip interface engaged with the radionuclide source clip from an unengaged configuration spaced from the radionuclide source holder to an engaged configuration to engage the radionuclide source clip with the radionuclide source holder at 530. Movement of the clip and associated interface from the unengaged configuration to the engaged configuration, in some embodiments, may include linearly translating the radionuclide source clip interface holding the radionuclide source clip in a first direction towards the adjacent portion of the radionuclide source holder. In some embodiments, the linear translation may be in a first vertical direction that is aligned with a direction of gravity. For instance, linear translation may proceed by depressing a plunger in direction D2 as shown in FIG. 5G. Moving the radionuclide source clip from a first unengaged configuration to an engaged configuration may further include compressing a gasket of the radionuclide source clip against a corresponding surface of the radionuclide source holder surrounding the radionuclide source to form a sealed volume with the radionuclide source holder and the radionuclide source clip. In some such embodiments, a radionuclide source may be contained in a radionuclide source receptacle of the holder. Additionally, engaging the radionuclide source clip with a distal end portion of the radionuclide source holder may include extending a portion of the radionuclide source clip into a radionuclide source receptacle of the radionuclide source holder to help maintain a position of a radionuclide source disposed in the source receptacle.

Method 500 may further include removing the radionuclide source holder engaged with the radionuclide source clip from the housing at 540. Removing the source holder may include moving the source holder in a second direction that may be substantially perpendicular to the first direction in which the linear translation of the radionuclide source clip interface moves when changing from an unengaged to engaged configuration. For instance, in some embodiments, the radionuclide source holder may be moved in a second direction as denoted with arrow D3 in FIG. 5H. As described above in the context of FIGS. 5C and 5D, the radionuclide source clip interface may include one or more detents configured to resist removal of the radionuclide source clip from the interface. However, once engaged with the radionuclide source holder, a force greater than the retaining force of the one or more detents resisting removal of the source clip may be applied to remove the clip and connected holder from the interface. Thus, when the source clip is engaged with the source holder, the clip may be removed from the housing with the source holder. In some such embodiments, when removing the radionuclide source holder from the housing, the radionuclide source clip that is selectively engaged with the holder may be slid along one or more rails supporting the clip (e.g., as shown in FIG. 5D) as the clip is removed from the housing with the holder, though other appropriate connections capable of supporting the clip thereon while permitting movement of the clip when engaged with the radionuclide source holder may be substituted.

Once the radionuclide source clip is engaged with the radionuclide source holder to maintain a position of the radionuclide source disposed therein, the method 500 may further include positioning the radionuclide source holder with the radionuclide source disposed therein into a transport container at 550. For example, this may include positioning the holder in a transport container 550 as shown in FIG. 2. Following positioning of the radionuclide source holder into a transport container, the radionuclide source holder may then be transported.

As described elsewhere herein, the method steps described above in the context of method 500 may be performed in the reverse to remove the clip from the radionuclide source holder. For instance, the method may include of inserting a distal end portion of a radionuclide source holder engaged with a radionuclide source clip into the housing such that the radionuclide source clip is engaged with the radionuclide source clip interface. The method may include moving the radionuclide source clip interface to disengage the radionuclide source clip form the radionuclide source holder. In some embodiments, the method includes changing the radionuclide source holder from an extended configuration to a retracted configuration. According to some embodiments, the method includes removing the radionuclide source holder, without the radionuclide source clip, from the housing. In some embodiments, the method may further comprise linearly translating the plunger in direction D2 to contain the radionuclide source clip within the housing of the handling system, as shown in the example embodiment depicted in FIG. 5I. Containing the radionuclide source clip within the housing of the handling system may advantageously prevent the escape of any radionuclides on a surface of the radionuclide source clip therefrom. In some embodiments, the method may further include disposing of the housing containing the radionuclide source clip.

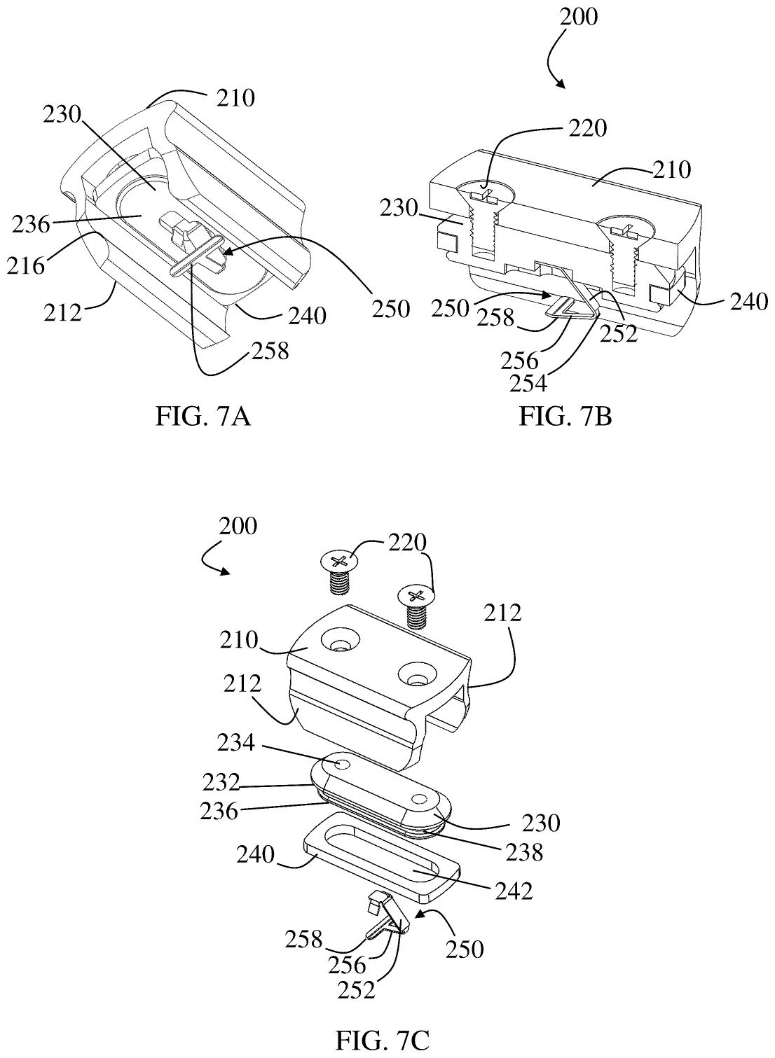

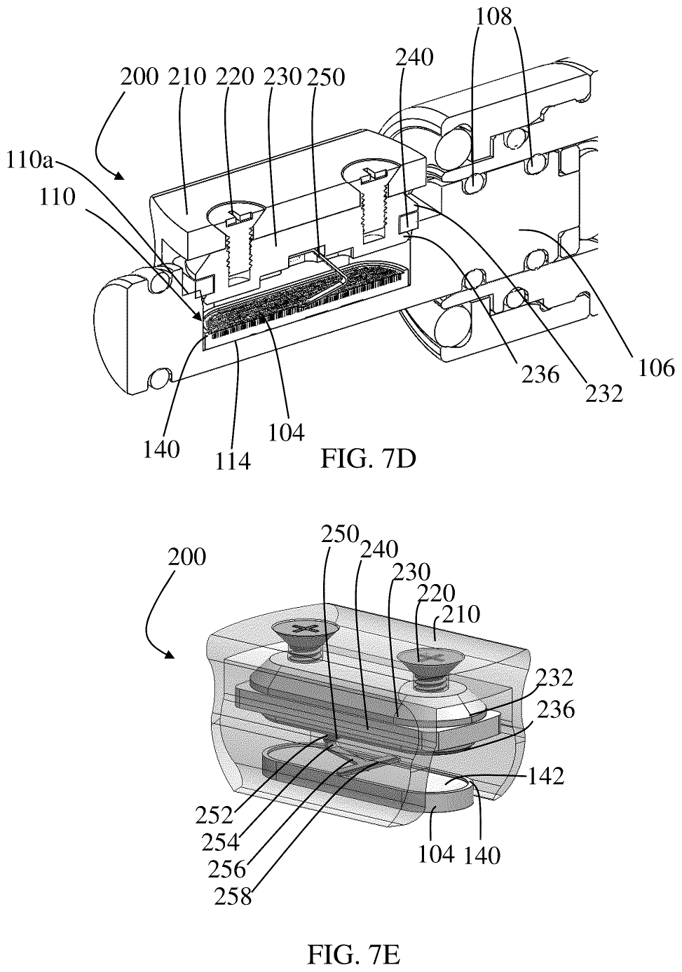

In some embodiments, it may be desirable to maintain a physical position and orientation of the radionuclide source 104 within a source receptacle 110 during transport, storage, and/or other instances where a radionuclide source 104 within a source holder 100 is not in use. In such an embodiment, the Inventors have recognized the benefits associated with the use of a radionuclide source clip 200 that is configured to be engaged with a radionuclide source holder 100 to maintain a position and/or orientation of a radionuclide source 104 within a receptacle 110 of the radionuclide source holder 100. Such a source may include gaskets, and attachment features similar to those described previously above as well as one or more protrusions that are configured to extend into the source receptacle 110 to contact the radionuclide source to maintain a desired position and orientation of the radionuclide source 104 as elaborated on further below. One such embodiment of a radionuclide source clip 200 is detailed further below relative to FIGS. 7A-7E.

Similar to the previously described embodiments, in the depicted embodiment, a radionuclide source clip 200 may include a clip body 210 and a gasket 240. In some embodiments, there may be a gasket holder 230 that may hold the gasket 240 relative to the clip body 210. Any appropriate fastener may be used to connect the gasket holder 230 to the clip body 210 between the two or more legs 212 of the clip 200. Alternatively, the gasket holder 230 may be integrally formed with, press fit into, and/or otherwise integrated with the clip body 210. The gasket 240 may be attached to and supported by the gasket holder 230 via a groove 238 that is sized and shaped to partially receive a portion of the gasket 240 therein. For example, similar to the prior embodiments, a second portion 236 of the gasket holder 230 may extend through and past an opening 242 of the gasket 240 into a channel extending longitudinally between the two opposing legs 212. In some embodiments the second portion 236 of the gasket holder 230 may extend in a direction that is oriented away from the clip body 210. In either case, the gasket 240 may be disposed at least partially in and extend outward from the groove 238 of the gasket holder 230. The groove 238 may completely or partly extend around a perimeter of the gasket holder. As noted previously, the gasket 240 may help form a seal between the source receptacle 110 and the source clip 200.

The clip 200 may also include other components that help promote sealing of a receptacle 110. For example, the source receptacle 110 may contain a sealing surface 110a surrounding the source receptacle 110 that further enables the gasket 240 of the source clip 200 to be sealed to the source receptacle 110. In some embodiments, the sealing surface 110a may be in the form of a flat planar surface that extends around the recess or other structure corresponding to the source receptacle 110. This sealing surface 110a may complement a shape of a corresponding surface of the gasket 240 to help facilitate the formation of a seal.

Similar to the other embodiments described herein, the two opposing legs 212 of the clip 200 may be deformable such that the legs 212 may be deformed at least partially around and be engaged with a portion of the rod 106 or other structure the source receptacle 110 is formed in or otherwise associated with. Again, the legs 212 may be configured to retain the clip 200 on the source holder 100 using latches including an angled surface 214 and a shelf 216 and/or any other appropriate type of locking structure. In either case, when the legs 212 secure the clip body 210 to the source holder 100, the clip 200 may be retained on the source holder 100 with the gasket 240 disposed between the clip body 210 and the source receptacle 110. For example, the gasket may be compressed between a portion of the clip 200, such as an upper first portion 232 of the gasket holder 230 and the sealing surface 110a surrounding the source receptacle 110 when the source clip 200 is attached to the source holder 100. The legs 212 may be constructed to apply a sufficient compressive force to the gasket 240 to generate a desired amount of sealing when engaged with the source holder 100.

As noted above, it may be desirable to maintain a position and orientation of a radionuclide source 104 within the source receptacle 110. Thus, in the depicted embodiment, the clip 200 may include one or more protrusions 250 that are configured to be engaged with the radionuclide source 104 when the clip 200 is engaged with the source receptacle 110 to maintain a position and orientation of the radionuclide source 104 disposed in the receptacle 110. For example, when the clip 200 is attached to the source holder 100, the one or more protrusions 250 may contact one or more portions of the radionuclide source 104 to prevent movement of the radionuclide source 104 in the source receptacle 110. Depending on the embodiment, the one or more protrusions may either be rigid and/or compliant elastic structures. For example, if it is desirable to apply a biasing force to the radionuclide source 104, then the one or more protrusions 250 may be configured to be compressed against the radionuclide source 104 to apply the desired biasing force to maintain the radionuclide source in the desired position and orientation.

In the depicted embodiment, the one or more protrusions may extend into the source receptacle 110 in a direction orientated at least partially away from the clip body 210 when engaged with the source holder 100. As the one or more protrusions 250 are configured to extend into the source receptacle 110 past the gasket 240, the one or more protrusions may also be positioned within and extend into the longitudinal channel extending between the two opposing legs 212. The one or more protrusions 250 may be attached to the clip body 210 in any appropriate fashion. For example, the one or more protrusions 250 may be integrally formed with or otherwise connected to the gasket holder 230. This may include mechanical attachments, adhesives, over molding, welding, fasteners, and/or any other appropriate type of attachment. In either case, the one or more protrusions 250 may be attached to the clip body 210 either directly or indirectly such that the one or more protrusions 250 may be configured to extend into the source receptacle 110 of a source holder 100 when the clip 200 is attached thereto.

As noted above, in some embodiments, the one or more protrusions 250 may be compliant such that they are deformed when biased against a radionuclide source 104. More specifically, in some embodiments, the one or more protrusions 250 may be elastic and compliant structures. Therefore, the protrusion 250 may be configured to elastically deform against the radionuclide source 104 when the clip 200 is attached to the source holder 100. To enable this elastic deformation, the one or more protrusions 250 may be fabricated from suitably clastic materials capable of being used for the desired durations in a radioactive environment while providing a desired biasing force to the radionuclide source 104. Appropriate materials may include, but are not limited to, elastic metals such as nitinol, titanium alloys, elastomers such as borated polyethylene, and/or any other appropriate material depending on the desired biasing force and/or the longevity of the protrusion 250. Alternatively, the one or more protrusions may be configured such that they are rigid and/or undergo a permanent deformation when compressed against the radionuclide source 104 as the disclosure is not so limited.

In some embodiments, the illustrated protrusion 250 is configured to function as a compressible spring. Specifically, in the depicted embodiment, the protrusion 250 includes separate first and second angled portions 252 and 256 formed from a flat metal structure that includes a living hinge 254 connected between the two angled portions 252 and 256. When compressed, the separate angled portions 252 and 256 and the living hinge 254 may be elastically deformed to apply the desired force. As shown in the figure, the first angled portion 252 may be connected to the gasket holder 230 and the second angled portion 256 may be configured to extend towards and be deformed against the radionuclide source 104. While a bent flat metal spring has been illustrated in the figures, it should be understood that other compliant structures with other shapes and constructions may also be used as the disclosure is not so limited. Additionally, it should be appreciated that rigid and/or permanently deformable compliant structures may also be used for the one or more protrusions.

During use, when a protrusion 250 is inserted into a source receptacle 110 of a source holder 100, the one or more protrusion 250 may be biased against a radionuclide source 104 that is disposed in the source receptacle 110, see FIGS. 7D-7E. Thus, the one or more protrusions 250 may apply a biasing force to the radionuclide source 104 to retain and/or prevent movement of the radionuclide source 104 in the source receptacle 110 of the source holder 100 when the clip 200 is engaged with the source holder 100. The applied force may be sufficient such that the radionuclide source 104 is held in a desired position and/or orientation against an inner wall 114 acting as a supporting surface of the source receptacle 110 the radionuclide source may be biased against when the clip 200 is engaged with the source holder 100. This force may hold the radionuclide source 104 in the desired position and orientation within the source receptacle 110 regardless of the source holder's orientation. As such, the protrusion 250 may limit or prevent movement of the radionuclide source relative to the source holder during transportation or storage.

In the above embodiments, the protrusion 250 may be a compliant, and in some instances an elastic component, that is deformed from a first extended position where the protrusion is not compressed to a second compressed position to apply a desired biasing force to a radionuclide source 104. However, in some embodiments, the protrusion 250 may be actively actuated from a first position to a second position after the clip 200 is secured to the source holder 100 by a user via an actuatable switch, button, slide, lever, actuator, or other appropriate control system configured to move the one or more protrusion 250 from a first position to a second position. In such an embodiment, the protrusion may be actuated to extend further into the source receptacle 110 to contact and hold the radionuclide source 104 in a desired position and orientation within the source receptacle 110.

During removal of the clip 200 from the source holder 100, the one or more protrusions 250 may be removed from within the source receptacle 110. In some embodiments, removing the one or more protrusions 250 may also result in the one or more protrusions 250 deforming back from the second position compressed against the radionuclide source 104 towards the first position which may correspond to an initial uncompressed state due to elastic recovery of the one or more protrusions 250. Alternatively, the one or more protrusions 250 may exhibit permanent deformation such that the one or more protrusions 250 do not revert back toward the undeformed configuration when removed.

While any appropriate force may be applied to a radionuclide source 104 by the one or more protrusions 250 of a radionuclide source clip, the selected range of forces may be selected to apply an appropriate biasing force to a radionuclide source 104 to maintain the radionuclide source 104 in a desired position and/or orientation while avoiding damage to the radionuclide source 104 and/or prevent the gasket from unsealing. For example, in some embodiments, the biasing force applied to a radionuclide source 104 by the one or more protrusions 250 of a radionuclide source clip 200 when engaged with a source holder 100 may be greater than or equal to 0.8 N, 0.9 N, 1.0 N, and 1.1 N. In some embodiments The biasing force may also be less than or equal to 1.2 N, 1.1 N, 1.0 N, and 0.9 N. Combinations of the forgoing are contemplated including a biasing force that is between or equal to 0.8 N and 1.2 N.

In some applications, it may be desirable to apply a biasing force at multiple locations and/or over a distributed surface area of a radionuclide source 104. For example, as best shown in FIGS. 7A and 7C, the illustrated protrusion 250 may include an interface 258 that is sized and shaped to distribute the force of the protrusion across a larger surface area and/or multiple locations of a radionuclide source 104 which may help to minimize or otherwise prevent damage to the radionuclide source 104. For example, the protrusion 250 includes an interface that is shaped as a cross bar that extends laterally out from an end portion of the protrusions, though other appropriately sized and shaped interfaces configured to contact one or more portions of the radionuclide source 104 may also be used. In some embodiments the bar may be sized and shaped to span the entire width and/or other appropriate dimension of the recess of the source receptacle 110. Alternatively, the interface 258 may be configured to span across only a portion of the width or other dimension of the source receptacle 110. Alternatively, or additionally, the interface may be sized to span across the entire (or close to the entire) width of the radionuclide source. In any case, this interface 258 may be sized and shaped to distribute the force over a larger area of the radionuclide source 104 than the attached end portion of the protrusion 250 would otherwise contact alone. For example, when the interface 258 is a bar, it may be configured to contact the radionuclide source 104 at two locations on opposing sides of the interface 258. In some embodiments, the interface 258 may contact the radionuclide source 104 on at least two, at least three, at least four, at least five and/or any number of other appropriate locations. As such, the interface may contact the radionuclide source 104 at any number of locations and may be formed with any appropriate geometry that allows the interface 258 to maintain the position and/or orientation of the radionuclide source 104 in the source holder 100.

As suggested above, the structure of the interface 258 may be sized and shaped to contact any one or more portions of the radionuclide source 104. However, it may be desirable to avoid direct contact between the interface and the active material of the radionuclide source 104 to avoid potential damage to the gaseous progeny radionuclide emitting surfaces and/or direct contact and contamination of the source with the interface 258. In some such embodiments, the radionuclide source 104 may include a substrate with an elevated portion 140 extending in an outward direction towards the interface 258 when the clip 200 is attached to the source holder 100. The elevated portion 140 may extend around an outer perimeter of the radionuclide source 104 with the precursor radionuclide contained in a recessed portion 142 within a central portion of the radionuclide source 104 disposed inward from the elevated portion 140. This elevated portion 140 may be part of a substrate or other non-radioactive material used to support the precursor radionuclide. Thus, the interface 258 may not directly contact the precursor radionuclide and/or the recessed portion 142 in such an embodiment. In the illustrated embodiment, the elevated portion 140 compliments a size and shape of the adjacent inner walls of the source receptacle 110.

When attached to a source holder, the interface 258 of the clip 200 may contact the elevated portion 140 of the radionuclide source 104 at one or more locations. For example, as best shown in FIG. 7E, in the depicted embodiment, the interface 258 may form a “T” shaped junction with the protrusion 250 that may extend away from the clip 200 and towards the radionuclide source 104. The spring like stem of the protrusion 250 with one or more bends along its length forms the stem of the “T” while the bar shape of the interface 258 forms the arm of the “T”, which may extend across the width of the radionuclide source 104 to contact the elevated portion 140 that may extend around a perimeter of the recessed portion 142 of the radionuclide source 104 containing the precursor radionuclide. The change in elevation from the elevated portion 140 to the recessed portion 142 may prevent the interface 258 from contacting the recessed portion 142 of the radionuclide source 104 and therefore, the clip 200 may only contact the radionuclide source 104 with the distal end portions (relative to the protrusion) of the interface 258 which may only contact the elevated portion 140 of the radionuclide source 104. Therefore, in embodiments where the interface 258 is spaced from the recessed portion 142 that contains the precursor radionuclide, the precursor radionuclide within the radionuclide source 104 may be disposed below and spaced apart from the interface 258 while the interface 258 contacts the elevated portion 140 of the source at two locations disposed on opposing sides of the longitudinal axis of the source 104. It should be appreciated that such a configuration may enable the interface 258 to minimize the points of contact with the radionuclide source 104 while still securing the source against the inner wall 114 of the source receptacle 110. Of course, interfaces 258 where a different number of locations or the entire perimeter of the radionuclide source 104 are contacted by the interface 258 and/or interfaces 258 that contact the precursor radionuclide may also be used as the disclosure is not so limited.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. A radionuclide source clip handling system, comprising:
a housing including an internal volume;
an opening formed in the housing, wherein the opening is configured to receive a distal end portion of a radionuclide source holder extending through the opening into the internal volume;
a radionuclide source clip interface disposed in the internal volume of the housing and configured to support a radionuclide source clip thereon, wherein the radionuclide source clip interface is moveable between an unengaged configuration and an engaged configuration, wherein the radionuclide source clip interface is configured to hold the radionuclide source clip spaced from the radionuclide source holder in the unengaged configuration when the radionuclide source holder is at least partially disposed in the opening, wherein the radionuclide source clip interface is configured to engage the radionuclide source clip with the distal end portion of the radionuclide source holder when the radionuclide source clip interface is in the engaged configuration and the radionuclide source holder is at least partially disposed in the opening.

2. The radionuclide source clip handling system of claim 1, wherein the radionuclide source clip interface is configured to permit the radionuclide source holder and the radionuclide source clip to be removed from the opening when the radionuclide source clip interface is in the engaged configuration.

3. The radionuclide source clip handling system of claim 1, wherein the radionuclide source clip interface includes one or more detents configured to resist removal of the radionuclide source clip from the radionuclide source clip interface.

4. The radionuclide source clip handling system of claim 1, wherein the radionuclide source clip interface is configured to linearly translate in the internal volume between the unengaged configuration and the engaged configuration.

5. The radionuclide source clip handling system of claim 1, wherein the radionuclide source clip interface is configured to translate in a first direction during movement between the unengaged configuration and the engaged configuration.

6. The radionuclide source clip handling system of claim 5, wherein the housing and the opening are configured to permit movement of the radionuclide source holder into and out of the opening in a second direction that is substantially perpendicular to the first direction.

7. The radionuclide source clip handling system of claim 1, wherein the radionuclide source clip interface is configured to slidingly support the radionuclide source clip.

8. The radionuclide source clip handling system of claim 1, wherein the radionuclide source clip interface includes one or more rails configured to slidingly support the radionuclide source clip thereon.

9. The radionuclide source clip handling system of claim 1, further comprising a plunger configured to move the radionuclide source clip interface between the unengaged configuration and the engaged configuration.

10. The radionuclide source clip handling system of claim 1, further comprising the radionuclide source clip disposed on the radionuclide source clip interface.

11. The radionuclide source clip handling system of claim 1, wherein the radionuclide source clip is configured to retain a radionuclide source within the distal end portion of the radionuclide source holder when the radionuclide source clip is engaged with the distal end portion of the radionuclide source holder.

12. The radionuclide source clip handling system of claim 1, further comprising the radionuclide source holder.

13. The radionuclide source clip handling system of claim 12, further comprising a radionuclide source disposed in the radionuclide source holder.

14. The radionuclide source clip handling system of claim 13, wherein the distal end portion of the radionuclide source holder comprises a radionuclide source receptacle configured to contain the radionuclide source, and wherein the radionuclide source clip interface is configured such that moving the radionuclide source clip to the engaged configuration places the radionuclide source clip over the radionuclide source in the radionuclide source receptacle.

15. The radionuclide source clip handling system of claim 14, wherein a portion of the radionuclide source clip is configured to extend into the radionuclide source receptacle to maintain a position of the radionuclide source in the radionuclide source receptacle.

16. The radionuclide source clip handling system of claim 15, wherein the radionuclide source clip includes a gasket configured to form a seal with the radionuclide source holder to isolate the radionuclide source when the radionuclide source clip is engaged with the radionuclide source holder.

17. A radionuclide source holder comprising:
a housing;
a radionuclide source receptacle;
a radionuclide source disposed in the radionuclide source receptacle; and
a radionuclide source clip disposed on and covering at least a portion of the radionuclide source receptacle, wherein the radionuclide source clip is configured to maintain the radionuclide source in the radionuclide source receptacle.

18. The radionuclide source holder of claim 17, wherein the radionuclide source receptacle is moveable between an extended configuration extending out from the housing and a retracted configuration disposed in the housing.

19. The radionuclide source holder of claim 17, wherein the radionuclide source clip extends at least partially into the radionuclide source receptacle.

20. The radionuclide source holder of claim 17, further comprising a gasket compressed between the radionuclide source clip and a radionuclide source clip interface to form a seal.

21. The radionuclide source holder of claim 17, wherein the radionuclide source clip includes two opposing legs configured to be engaged with and retain the radionuclide source clip on the radionuclide source receptacle.

22. The radionuclide source holder of claim 21, wherein the radionuclide source clip includes grooves formed in the two opposing legs, wherein the grooves extend along a longitudinal length of the two opposing legs.

23. A method of attaching a radionuclide source clip interface, comprising:
inserting at least a portion of a radionuclide source holder into an internal volume of a housing;
moving the radionuclide source clip interface engaged with a radionuclide source clip from an unengaged configuration spaced from the radionuclide source holder to an engaged configuration to engage the radionuclide source clip with the radionuclide source holder; and
removing the radionuclide source holder engaged with the radionuclide source clip from the radionuclide source clip interface and the housing.

24. The method of claim 23, wherein inserting at least a portion of the radionuclide source holder comprises inserting at least a distal end portion of the radionuclide source holder.

25. The method of claim 23, further comprising using a plunger to move the radionuclide source clip interface from the unengaged configuration to the engaged configuration.

26. The method of claim 23, wherein attaching the radionuclide source clip to the radionuclide source holder comprises attaching the radionuclide source clip to a radionuclide source receptacle of the radionuclide source holder.

27. The method of claim 23, further comprising changing the radionuclide source holder from a retracted configuration wherein a radionuclide source is not exposed to an ambient atmosphere to an extended configuration wherein the radionuclide source is exposed to the ambient atmosphere.

28. The method of claim 27, further comprising engaging the radionuclide source clip with a distal end portion of the radionuclide source holder.

29. The method of claim 28, further comprising sealing the radionuclide source in the radionuclide source holder with the radionuclide source clip.

30. The method of claim 27, further comprising linearly translating the radionuclide source clip interface engaged with the radionuclide source clip in a first direction within an interior volume of the housing to move the radionuclide source clip from the unengaged configuration to the engaged configuration.

31. The method of claim 30, wherein removing the radionuclide source holder engaged with the radionuclide source clip from the housing comprises translating the radionuclide source holder in a second direction that is substantially perpendicular to the first direction.

32. The method of claim 23, further comprising transporting the radionuclide source holder engaged with the radionuclide source clip.

33. The method of claim 23, wherein moving the radionuclide source clip interface engaged with the radionuclide source clip comprises depressing a plunger configured to move the radionuclide source clip interface.

34. The method of claim 23, further comprising extending a portion of the radionuclide source clip into a radionuclide source receptacle of the radionuclide source holder to maintain a position of a radionuclide source disposed in the radionuclide source receptacle.

35. A radionuclide source clip comprising:
a clip body;
two opposing legs extending away from the clip body and configured to be engaged with a portion of a radionuclide source holder and retain the radionuclide source clip on a source receptacle of the radionuclide source holder;
a gasket connected to the clip body between the two opposing legs; and
a protrusion connected to the clip body and disposed between the two opposing legs, wherein the protrusion extends in a direction orientated at least partially away from the clip body.

36. The radionuclide source clip of claim 35, wherein the protrusion extends past the gasket into a channel extending longitudinally between the two opposing legs.

37. The radionuclide source clip of claim 36, wherein the protrusion is a compliant elastic protrusion.

38. The radionuclide source clip of claim 36, wherein the protrusion is configured to maintain a position and orientation of a radionuclide source disposed within the source receptacle when the radionuclide source clip is attached to the radionuclide source holder.

39. The radionuclide source clip of claim 38, wherein the protrusion includes an interface configured to contact one or more elevated portions of the radionuclide source disposed in the source receptacle and be spaced from a recessed portion of the radionuclide source containing a precursor radionuclide.

40. The radionuclide source clip of claim 35, further comprising a gasket holder connected to the clip body between the two opposing legs, wherein the gasket holder includes a groove extending around a perimeter of the gasket holder, and wherein the gasket is disposed at least partially within the groove.

41. The radionuclide source clip of claim 40, wherein the gasket includes an opening, and a portion of the gasket holder extends through the opening in a direction directed away from the clip body.

42. The radionuclide source clip of claim 41, wherein the protrusion is connected to and extends away from the gasket holder.

43. The radionuclide source clip of claim 39, wherein the gasket is configured to be disposed between the clip body and the source receptacle when the radionuclide source clip is attached to the radionuclide source holder.

44. A method of handling a radionuclide source, the method comprising:
attaching a radionuclide source clip to a source receptacle of a radionuclide source holder, wherein the radionuclide source is disposed in the source receptacle;
compressing a gasket of the radionuclide source clip against a portion of the radionuclide source holder surrounding the source receptacle to seal the source receptacle; and
maintaining a pose of the radionuclide source in the source receptacle with a protrusion of the radionuclide source clip extending into the source receptacle and in contact with the radionuclide source.

45. The method of claim 44, wherein attaching the radionuclide source clip includes engaging the radionuclide source holder with two opposing legs of the radionuclide source clip.

46. The method of claim 45, wherein the protrusion extends into a channel.

47. The method of 46, further comprising elastically deforming the protrusion to apply a biasing force on the radionuclide source.

48. The method of claim 47, further comprising contacting one or more elevated portions of the radionuclide source with an interface of the protrusion, and wherein the interface is spaced from a recessed portion of the radionuclide source containing a precursor radionuclide.

49. The method of claim 44, wherein the gasket includes an opening, and a portion of a gasket holder extends through the opening in a direction directed away from a clip body of the radionuclide source clip.

50. The method of claim 49, wherein attaching the radionuclide source clip to the source receptacle compresses the protrusion against the radionuclide source.

51. The method of claim 49, further comprising compressing the gasket between the clip body and the source receptacle.
